(19) United States
(12) Patent Application Publication
(10) Pub. No.: US 2023/0016674 A1
(43) Pub. Date: Jan. 19, 2023

(54) Method for obtaining chitin and/or chitosan using two enzymatic hydrolyses

(71) Applicant: YNSECT, Évry-Courcouronnes Cedex (FR)

(72) Inventors: Lorena SANCHEZ, JUVISY-SUR-ORGE (FR); Valère HUSSET, LES CLAYES SOUS BOIS (FR); Adam KIRECHE, CHAMPIGNY-SUR-MARNE (FR)

(73) Assignee: YNSECT, Évry-Courcouronnes Cedex (FR)

(21) Appl. No.: 17/782,236

(22) PCT Filed: Dec. 17, 2020
(86) PCT No.: PCT/FR2020/052517

§ 371 Date: Jun. 3, 2022

(30) Foreign Application Priority Data
Dec. 19, 2019 (FR) 1915001

(51) Int. Cl. C12P 19/26; C12N 9/50; C08B 37/08

(52) U.S. Cl. CPC C12P 19/26; C12N 9/50; C12Y 304/21062; C12Y 304/24028; C08B 37/003

(57) ABSTRACT

The present invention relates to a method for obtaining chitin and/or chitosan from insect cuticles. More particularly, the method according to the present invention comprises a first enzymatic hydrolysis of insect cuticles using at least one endopeptidase, separation from the hydrolysis medium of the hydrolyzed cuticles resulting from the first enzymatic hydrolysis, and a second enzymatic hydrolysis of the hydrolyzed cuticles using at least one endopeptidase, excluding exopeptidase.

The present invention relates to a method for obtaining chitin from insect cuticles.

By “chitin” is meant a polymer composed mainly of glucosamine and N-acetyl-glucosamine units, said units being optionally substituted by amino acids and/or peptides.

It is known that chitin is synthesized by many species of the living world, and in particular by insects since it constitutes 3 to 60% of their exoskeleton.

The cuticle is the outer layer (or exoskeleton) secreted by the epidermis of the insects. It is generally formed of three layers: the epicuticle, the exocuticle and the endocuticle.

Insects are a renewable and abundant raw material, so it is very advantageous to extract chitin from them.

Chitin is also known to be a source of chitosan, obtained after deacetylation thereof.

By “chitosan” is meant according to the present invention the deacetylation products of chitin. The usual limit between chitosan and chitin is determined by the degree of acetylation: a compound with a degree of acetylation of less than 50% is called chitosan, beyond that, a compound with a degree of acetylation of more than 50% is called chitin.

Chitin and chitosan have many applications, particularly in the food, cosmetics or medical fields.

Chitin is usually obtained by a chemical or enzymatic method, the purpose of these methods being to eliminate the proteins bound to the chitin. The use of an enzymatic method is especially advantageous since in particular it allows the structure of the chitin to be kept as close as possible to its original structure.

However, the enzymatic methods usually used involve a long reaction time and do not allow chitin to be deproteinized satisfactorily.

The work of the inventors has made it possible to develop an enzymatic extraction method that overcomes these drawbacks.

Thus, the present invention relates to a method for obtaining chitin from insect cuticles comprising:

performing a first enzymatic hydrolysis of insect cuticles using at least one endopeptidase,
separating the hydrolyzed cuticles resulting from the first enzymatic hydrolysis, from the hydrolysis medium,
performing a second enzymatic hydrolysis of the hydrolyzed cuticles using at least one endopeptidase, excluding exopeptidase.

By “insects” is meant insects at any stage of development, such as an adult, larval or nymph stage.

Advantageously, the insects preferred according to the invention are Coleoptera, Diptera, Lepidoptera, Orthoptera, Hymenoptera, Dictyoptera to which belong in particular Blattoptera, included therein being Isoptera and Mantoptera, Phasmoptera, Hemiptera, Heteroptera, Ephemeroptera and Mecoptera, or mixtures thereof, preferably Coleoptera, Diptera, Lepidoptera, Orthoptera or mixtures thereof, more preferably Coleoptera.

Preferentially, the Diptera belong to the Brachycera suborder.

Preferentially, the Lepidoptera belong to the Ditrysia suborder, more preferentially to the Pyraloidea superfamily.

Preferably, the Coleoptera belong to the infra-order Cucujiformia, in particular to the families Tenebrionidae, Coccinellidae, Cerambycidae, Dryophthoridae or mixtures thereof.

More preferably, the Coleoptera are chosen from Tenebrio molitor, Alphitobius diaperinus, Zophobas morio, Tenebrio obscurus, Tribolium castaneum, Rhynchophorus ferrugineus and mixtures thereof, still more preferably Tenebrio molitor.

Advantageously, the insect cuticles are obtained from the larval stage of the aforementioned insect species.

By “enzymatic hydrolysis” is meant a chemical reaction in which a chemical bond is broken by water, this breakage being catalyzed by an enzyme or enzyme composition.

According to the invention, the enzyme or enzyme composition has proteolytic (protease) activity.

By “peptidase” or “protease” is meant an enzyme capable of cleaving a peptide bond of a peptide chain.

The names or suffixes “protease” and “peptidase” are used interchangeably throughout the present application.

By “endopeptidase” is meant a peptidase capable of cleaving a peptide bond internal to a peptide chain, and which therefore acts preferably away from a terminal peptide bond of a peptide chain (i.e. located at a C- or N-terminus of the peptide chain).

By “exopeptidase” is meant an enzyme whose mechanism of action makes it possible to cleave only a terminal peptide bond of a peptide chain, leading to the release of an amino acid, a dipeptide or a tripeptide.

Sometimes commercially available enzymes are not pure. The choice of an enzyme thus implies the presence of other enzymes (in lesser amount) having the same type of activity (for example other types of endopeptidase) or even different activities (for example exopeptidases, lipases).

Therefore, according to the invention, by “endopeptidase” or “exopeptidase”, it is meant that the main enzyme is, respectively, an endopeptidase or an exopeptidase. By “main enzyme” is meant the enzyme whose activity is sought. More particularly, this activity is specified by the manufacturer in the marketed product. This specification can be made by an indication in the data sheets (safety, marketing) associated with the product.

It may also be the case that the product is an enzyme composition, i.e. a mixture of several main enzymes. In such case, each of the enzymes is specified by the manufacturer in the marketed product.

Advantageously, the at least one endopeptidase used in the first and second enzymatic hydrolysis of the method according to the invention is an enzyme or a proteolytic enzyme composition, identical or different, the presence of main enzymes (specified by the manufacturer) other than proteolytic being excluded, in order to promote proteolytic activity.

Advantageously, the at least one endopeptidase used in the first enzymatic hydrolysis of the method according to the invention is selected from an endopeptidase, a mixture of endopeptidases and a mixture of exopeptidase(s) and endopeptidase(s), preferably from an endopeptidase and a mixture of endopeptidases, and more preferably is an endopeptidase.

Advantageously, the at least one endopeptidase used in the first hydrolysis is selected from serine endopeptidases (EC/IUB 3.4.21.XX), cysteine endopeptidases (EC/IUB 3.4.22.XX), aspartic proteases (EC/IUB 3.4.23.XX), metalloendopeptidases (EC/IUB 3.4.24.XX) and threonine endopeptidases (EC/IUB 3.4.25.XX).

Preferably, the at least one endopeptidase used in the first hydrolysis is selected from serine endopeptidases, cysteine endopeptidases, aspartic proteases and metalloendopeptidases, more preferably, serine endopeptidases and metalloendopeptidases, even more preferably serine endopeptidases.

When the at least one endopeptidase, used in the first hydrolysis only, is an enzyme composition in the form of a mixture of exopeptidase(s) and endopeptidase(s), the exopeptidase(s) is/are selected from aminopeptidases (EC/IUB 3.4.11.XX), dipeptidases (EC/IUB 3.4.13.XX), di- or tripeptidyl peptidases (EC/IUB 3.4.14.XX), peptidyl dipeptidases (EC/IUB 3.4.15.XX), serine carboxypeptidases (EC/IUB 3.4.16.XX), metallocarboxypeptidases (EC/IUB 3.4.17.XX), cysteine carboxypeptidases (EC/IUB 3.4.18.XX) and omega peptidases (EC/IUB 3.4.19.XX).

Preferably, the exopeptidase(s) that can be used in the first hydrolysis is (are) selected from aminopeptidases, di- or tripeptidyl peptidases and serine carboxypeptidases, more preferably aminopeptidases.

By way of example, in the first enzymatic hydrolysis, when the at least one endopeptidase is an enzyme composition, which is a mixture of exopeptidase(s) and endopeptidase(s), the endopeptidase is as indicated above.

Preferably, the at least one endopeptidase is then a mixture of a serine endopeptidase, an aspartic protease and an aminopeptidase.

By “separation from the hydrolysis medium”, is meant more particularly the isolation and recovery of the hydrolyzed cuticles.

Advantageously, the separation of the hydrolyzed cuticles resulting from the first enzymatic hydrolysis is performed by centrifugation, decantation or filtration, preferably by filtration.

Advantageously, this filtration is carried out using a Büchner filter, a filter press, a belt filter or a sieve filter, preferably using a Büchner filter with a metal mesh. Preferably, the pore size or mesh size of the filter used in this filtration is between 4 μm and 800 μm, more preferably between 40 μm and 550 μm, even more preferably between 140 μm and 530 μm, for example such as 160 μm.

It will be noted that, in the context of the present application, and unless otherwise specified, the ranges of values indicated are understood to be inclusive.

It is understood that during the separation of the hydrolyzed cuticles, the at least one endopeptidase (as an enzyme or enzyme composition) used in the first enzymatic hydrolysis is discharged with the hydrolysis medium; it is therefore not or very little present during the second enzymatic hydrolysis.

The second enzymatic hydrolysis uses at least one endopeptidase, excluding exopeptidase.

Similarly, by “at least one endopeptidase, excluding exopeptidase” is meant an endopeptidase as defined above, on condition that the second hydrolysis does not comprise exopeptidase, i.e. that the at least one endopeptidase is an endopeptidase or is an enzyme composition not comprising exopeptidase.

Preferably, the at least one endopeptidase involved in this second enzymatic hydrolysis is an endopeptidase or a mixture of endopeptidases, more preferably an endopeptidase.

More particularly, in the method according to the invention, the at least one endopeptidase used in the second enzymatic hydrolysis is an endopeptidase or a mixture of endopeptidases selected from serine endopeptidases, cysteine endopeptidases, aspartic proteases, metalloendopeptidases and threonine endopeptidases.

Preferably, the at least one endopeptidase used in the second enzymatic hydrolysis is an endopeptidase or a mixture of endopeptidases selected from serine endopeptidases, cysteine endopeptidases, aspartic proteases and metalloendopeptidases.

More preferably, the at least one endopeptidase used in the second enzymatic hydrolysis is an endopeptidase or a mixture of endopeptidases selected from serine endopeptidases and metalloendopeptidases.

In particular, in the method according to the invention, the at least one endopeptidase used in the second enzymatic hydrolysis is a serine endopeptidase or a mixture of serine endopeptidases, and/or a metalloendoprotease or a mixture of metalloendoproteases, each serine endopeptidase being selected from the group constituted by subtilisin and trypsin, each metalloendoprotease being selected from the group constituted by bacillolysin and thermolysin.

More particularly, the at least one endopeptidase used in the second enzymatic hydrolysis is more preferably a serine endopeptidase and/or a metalloendoprotease, the serine endopeptidase being subtilisin, and the metalloendoprotease being bacillolysin.

Lastly, still more preferably, the at least one endopeptidase used in the second enzymatic hydrolysis is an endopeptidase selected from the serine endopeptidases.

The endopeptidases used in the first and the second hydrolysis step may be identical, for example such as a same serine endopeptidase or a same metalloendoprotease.

According to a particular embodiment, the at least one endopeptidase used in the first enzymatic hydrolysis comprises a serine endopeptidase, and is preferably a serine endopeptidase, and the at least one endopeptidase used in the second enzymatic hydrolysis is also a serine endopeptidase, the group of the serine endopeptidases being advantageously as described below.

In the method according to the invention, the serine endopeptidases used in any of the hydrolysis steps are advantageously selected from the group constituted by subtilisin (EC/IUB 3.4.21.62), chymotrypsin (EC/IUB 3.4.21.1), trypsin (EC/IUB 3.4.21.4), oryzin (EC/IUB 3.4.21.63), glutamyl endopeptidase (EC/IUB 3.4.21.19), streptogrisin A (EC/IUB 3.4.21.80) and/or streptogrisin B (EC/IUB 3.4.21.81), preferably subtilisin, trypsin and/or oryzin, more preferably, subtilisin and/or trypsin, even more preferably, subtilisin

Similarly, the cysteine endopeptidases used in any of the hydrolysis steps are advantageously selected from the group constituted by bromelain (EC/IUB 3.4.22.32) and/or papain (EC/IUB 3.4.22.2), preferably bromelain.

Similarly, the aspartic proteases used in any of the hydrolysis steps are advantageously aspergillopepsin I (EC/IUB 3.4.23.18).

Similarly, the metalloendoproteases used in any of the hydrolysis steps are advantageously selected from the group constituted by bacillolysin (EC/IUB 3.4.24.28), thermolysin (EC/IUB 3.4.24.27), collagenase I (EC/IUB 3.4.24.3), vibriolysin (EC/IUB 3.4.24.25), pseudolysin (EC/IUB 3.4.24.26), aureolysin (EC/IUB 3.4.24.29), coccolysin (EC/IUB 3.4.24.30), mycolysin (EC/IUB 3.4.24.31), beta-lytic metalloendopeptidase (EC/IUB 3.4.24.32), deuterolysin (EC/IUB 3.4.24.39) and/or serralysin (EC/IUB 3.4.24.40), preferably bacillolysin, thermolysin and/or collagenase I, more preferably bacillolysin and/or thermolysin, still more preferably bacillolysin.

Advantageously, the aminopeptidases used in the first hydrolysis step are leucyl aminopeptidase (EC/IUB 3.4.11.1).

Advantageously, the at least one endopeptidase used in any of the enzymatic hydrolysis steps of the method according to the invention is of bacterial, fungal, animal and/or plant origin, preferably of bacterial and/or fungal origin, more preferably of bacterial origin.

By way of example, the at least one endopeptidase of plant origin is bromelain or papain, preferably bromelain. In particular, the at least one endopeptidase of plant origin may come from Ananas comosus or Carica papaia.

By way of example, the at least one endopeptidase of animal origin is trypsin and/or chymotrypsin, more preferably trypsin. In particular, the at least one endopeptidase of animal origin may come from pig pancreases (sus scrofa domesticus).

By way of example, the at least one endopeptidase of fungal origin is oryzin and/or leucyl aminopeptidase and/or aspergillopepsin I, preferably oryzin or a mixture of oryzin, leucyl aminopeptidase and aspergillopepsin I, more preferably, a mixture of oryzin, leucyl aminopeptidase and aspergillopepsin I. In particular, the at least one endopeptidase of fungal origin may come from Aspergillus, more particularly Aspergillus oryzae or Streptomyces, more particularly Streptomyces griseus, preferably from Aspergillus oryzae.

By way of example, the at least one endopeptidase of bacterial origin is subtilisin, bacillolysin, thermolysin, collagenase I or a mixture thereof, preferably subtilisin and/or bacillolysin, more preferably subtilisin. In particular, the at least one endopeptidase of bacterial origin may come from Bacillus or Clostridium histolyticum, preferably from Bacillus licheniformis, Bacillus subtilis, Bacillus amyloliquefaciens or Bacillus stearothermophilus, more preferably from Bacillus licheniformis.

In particular, in the method according to the invention, the at least one endopeptidase used in the second enzymatic hydrolysis is of bacterial origin.

Advantageously, the enzymes or enzyme composition of the method according to the invention are selected from the marketed enzymes of the following Table 1:

TABLE 1

| Trade name | Origin | Species | Enzyme | Supplier |
|---|---|---|---|---|
| Promod | Bacterial | Bacillus | Subtilisin (CAS 9014- | Biocatalysts |
| 439L | | licheniformis | 01-1)*, EC/IUB | |
| | | | 3.4.21.62 | |
| Novo-Pro D | | | Subtilisin*, EC/IUB | Novozymes |
| | | | 3.4.21.62* | |
| Novozym | | | Subtilisin*, EC/IUB | Novozymes |
| 37071 | | | 3.4.21.62* | |
| Alcalase 2.5 | | | Subtilisin*, EC/IUB | Novozymes |
| L PF | | | 3.4.21.62* | |
| Corolase | | Bacillus subtilis | Metallo et serine | AB Enzymes |
| 7089 | | | protéase* | |
| | | | EC/IUB 3.4.2X.XX* | |
| Sumizyme | | | EC/IUB 3.4.2X.XX* | Shin Nihon |
| BNP-L | | | | Chemical |
| Food Pro | | Bacillus | Bacillolysin*, EC/IUB | Dupont |
| PNL | | amyloliquefaciens | 3.4.24.28 | Danisco |
| Protin SD- | | | Bacillolysin, EC/IUB | Amano Enzyme |
| NY100 | | | 3.4.24.28* | |
| Corolase | | Bacillus | Thermolysin, EC/IUB | AB Enzymes |
| 2TS | | stearothermophilus | 3.4.24.27* | |
| Collagenase | | Clostridium | Collagenase (CAS | Alfa Aesar |
| Type I | | histolyticum | 9001-12-1)*, EC/IUB | |
| | | | 3.4.24.3 | |
| Promod | Bacterial and | Mixture of strains, | Subtilisin, (CAS 9014- | Biocatalysts |
| 648L | plant | mainly Bacillus | 01-1)*, EC/IUB | |
| | | | 3.4.21.62; | |
| | | | Papain, (CAS 9001- | |
| | | | 73-4)*, EC/IUB | |
| | | | 3.4.22.2 | |
| Prolyve NP | Fongique | Aspergillus oryzae | Oryzin, EC/IUB | Soufflet |
| conc | | | 3.4.21.63* | Biotechnologies |
| FoodPro | | | Aspergillopepsin I*, | Dupont |
| 51FP | | | EC/IUB 3.4.23.18 | Danisco |
| Sumizyme | | | Leucyl | Shin Nihon |
| LP | | | aminopeptidase, | Chemical |
| | | | EC/IUB 3.4.11.1*; | |
| | | | Oryzin, EC/IUB | |
| | | | 3.4.21.63*; | |
| | | | Aspergillopepsin I, | |
| | | | EC/IUB 3.4.23.18* | |
| Protease | | Streptomyces | S. griseus Protease | Sigma Aldrich |
| type XIV | | griseus | A*, EC/IUB 3.4.21.80; | |
| | | | S. griseus Protease | |
| | | | B*. EC/IUB 3.4.21.81; | |
| | | | S. griseus Trypsin*; | |
| | | | Aminopeptidase* | |
| Pancreatin | Animal | Sus scrofa | Trypsin*, EC/IUB | Sigma-Aldrich |
| | | domesticus | 3.4.21.4; | |
| | | | Amylase*; | |
| | | | Lipase*; | |
| | | | Ribonuclease*; | |
| | | | Protease* | |
| Bromelain | Plant | Ananas comosus | Bromelain, EC/IUB | Sigma-Aldrich |
| from | | | 3.4.22.32* | |
| pineapple | | | | |
| stem | | | | |
| Lypaïne | | Carica papaïa | Papain, EC/IUB | Soufflet |
| 48000 | | | 3.4.22.2* | Biotechnologies |

*Stated by the supplier

Preferably, the at least one endopeptidase used in the first hydrolysis of the method according to the invention is selected from Promod 439L, Novo-Pro D, Novozym 37071, Alcalase 2.5 L PF and Sumizyme LP, more preferably, Promod 439L, Alcalase 2.5 L PF and Sumizyme LP.

Preferably, the at least one endopeptidase used in the second hydrolysis of the method according to the invention is selected from Promod 439L, Novo-Pro D, Novozym 37071 and Alcalase 2.5 L PF, more preferably, Promod 439L and Alcalase 2.5 L PF.

According to a particularly preferred embodiment, the at least one endopeptidase used in the first enzymatic hydrolysis is of bacterial or fungal origin, and the at least one endopeptidase used in the second enzymatic hydrolysis is of bacterial origin.

In this embodiment, each at least one endopeptidase used in the hydrolysis steps is advantageously one of those described above.

Thus, according to this embodiment, the at least one endopeptidase used in the first enzymatic hydrolysis is preferably subtilisin, or a mixture of oryzin, leucyl aminopeptidase and aspergillopepsin I, or bacillolysin, more preferably subtilisin, and the at least one endopeptidase used in the second enzymatic hydrolysis is preferably subtilisin or bacillolysin, more preferably subtilisin.

A thermal inactivation of the at least one endopeptidase (enzyme or enzyme composition) used in the hydrolysis steps of the method according to the invention can be carried out following any of the enzymatic hydrolysis steps. Advantageously, only one thermal inactivation is carried out, following the second enzymatic hydrolysis.

Preferably, this thermal inactivation is performed by heating the hydrolysis medium to between 80° C. and 105° C., for example such as 85° C., for 10 to 30 minutes, more preferably, for 15 to 20 minutes.

Advantageously, the reaction medium obtained at the end of the second enzymatic hydrolysis of the method according to the invention is heated to a temperature greater than 70° C., preferably greater than 75° C., more preferably greater than 80° C., for example such as 85° C., in order to inactivate the at least one endopeptidase.

Advantageously, the method according to the invention further comprises a step of grinding the insect cuticles prior to the first enzymatic hydrolysis.

Preferably, the insect cuticles are ground in the presence of water, more preferably in a weight ratio of cuticles to water comprised between 1:5 and 1:7, for example such as 1:6.

Throughout the present application, water is preferably tap water.

Preferably, the grinding step makes it possible to reduce the size of the insect cuticles to a size comprised between 0.5 mm and 10 mm, more preferably, between 1.0 and 7 mm, still more preferably, between 1.0 and 5 mm, for example such as between 1.0 and 2.0 mm.

This grinding step in particular makes it possible to improve the efficiency of the hydrolysis steps and thus, to reduce the duration of these steps.

Advantageously, the grinding step of the insect cuticles is carried out with a meat grinder, hammer mill, blade mill, knife mill and/or disperser, preferably with a knife mill such as a Thermomix.

Advantageously, the insect cuticles are washed with water after the grinding step and before the first enzymatic hydrolysis. This step improves the removal of proteins that are not bound to the cuticles.

Advantageously, these washed insect cuticles are collected by filtration, preferably using a Büchner filter, a filter press, a belt filter or a sieve filter, preferably using a Büchner filter with a metal mesh.

Preferably, the pore or mesh size of this filter is between 4 μm and 800 μm, more preferably, between 40 μm and 550 μm, still more preferably between 140 μm and 530 μm.

Advantageously, the washed cuticles are pressed after their collection by a filtration step in order to remove excess water. Preferably, said cuticles are pressed to attain a dry matter content comprised between 35 and 65% by weight, more preferably, between 45 and 55% by weight, with respect to the total weight of the cuticles.

The skilled person knows which type of press to use for this pressing step, for example the Angelia 7500 press from Angel.

Pressed insect cuticles may be frozen before the first enzymatic hydrolysis step.

Advantageously, the insect cuticles of the method according to the invention are obtained by a step of separating the cuticles from the soft part of the insects.

By “soft part” is meant the flesh (comprising in particular the muscles and the viscera) and the juice (comprising in particular the body fluids, water and haemolymph) of the insects. In particular, the soft part does not consist of the juice of the insects.

This step of separating the cuticles from the soft part is generally preceded by a step of killing the insects, which can advantageously be carried out by thermal shock, such as by scalding or by blanching.

For scalding, the insects, preferably larvae, are thus scalded with water for 2 to 20 minutes, preferably 5 to 15 minutes. Preferably, the water is at a temperature of 87 to 100° C., preferably 92 to 95° C.

The amount of water added in the scalding is determined as follows: the ratio of the volume of water in ml to the weight in g of insect is preferably comprised between 0.3 and 10, more preferentially between 0.5 and 5, even more preferentially between 0.7 and 3, and is still more preferentially of the order of 1.

For the blanching, the insects, preferably larvae, are blanched with water or with steam (steam nozzles or bed) at a temperature comprised between 80 and 105° C., preferably between 87 and 105° C., more preferentially between 95 and 100° C., still more preferentially 98° C. or with water at a temperature comprised between 90 and 100° C., preferentially between 92 and 95° C. (by spray nozzles) or in mixed mode (water+steam) at a temperature comprised between 80 and 130° C., preferably between 90 and 120° C., more preferentially between 95 and 105° C., still more preferentially 98° C. When the insects are blanched with steam only, the blanching is advantageously carried out in forced steaming blanching machines. The residence time in the bleaching chamber is comprised between 5 seconds and 15 minutes, preferably between 1 and 7 minutes.

Advantageously, following the killing step, the insects are directly used for the implementation of the step of separating the cuticles from the soft part of the insects, i.e. the insects are not subjected to any treatment such as grinding, freezing or dehydration, between these two steps.

Preferably, this step of separating the cuticles from the soft part of the insects is carried out with a filter press or a belt separator, more preferably with a belt separator.

The term “belt separator” refers to a device that has a squeezing belt (or press belt) and a perforated drum.

The squeezing belt allows the insects to be conveyed to and applied against the perforated drum so as to cause the soft part of the insects to pass, by pressure, through the perforations of the drum, while the cuticles remain outside the drum.

The cuticles can then be recovered with a scraper knife and optionally frozen.

By way of example there may be mentioned the belt separators from Baader, such as the belt separators 601 to 607 (“soft separator 601 to 607”) or for instance the SEPAmatic® belt separators from BFD Corporation (410 to 4000 V range).

Advantageously, the diameter of the perforations of the drum is comprised between 0.5 and 3 mm, preferably between 1 and 2 mm.

As regards the pressure, the person skilled in the art is capable of determining the pressure to be applied, enabling the separation of the cuticles from the soft part of the insects.

This step of separation of the insects is different from conventional pressing that can be carried out for example with a single-screw or twin-screw press in that it allows a (clean) separation of the soft part and the cuticles of the insects and not a separation of a juice from a solid fraction.

In particular, the separation of the cuticles from the soft part is carried out without performing any prior grinding step of the insects, especially in the form of particles.

Advantageously, the total time of the hydrolysis steps of the method according to the invention is less than 6 h.

By “total time of the hydrolysis steps” is meant the sum of the reaction times of the first and the second hydrolysis step.

Typically, for each hydrolysis, the hydrolysis reaction time can be defined as the time elapsed from the addition of the at least one endopeptidase to the hydrolysis medium to the step of separating the hydrolysis medium from the hydrolyzed cuticles, or inactivation of the hydrolysis medium.

Preferably, the total time of the hydrolysis steps is between 2 h and 5 h 30, more preferably, between 3 h and 5 h, even more preferably 4 h.

Advantageously, the duration of each hydrolysis step is between 30 min and 4 h, preferably between 1 h and 3 h, more preferably between 1 h 30 and 2 h 30, for example such as equal to 2 h.

Advantageously, the total amount of said at least one endopeptidase used in the method according to the invention is between 10 U and 500 U per gram of insect cuticle, on a dry weight basis.

It is understood that throughout the text of the application, “U”, also referred to as an enzyme unit, represents the amount of enzyme required to release one micromole of tyrosine (from casein) in one minute at 37° C. and pH 7.5.

By “total amount of said at least one endopeptidase” is meant the sum of the amounts of the at least one endopeptidase introduced in the first and second enzymatic hydrolysis of the method according to the invention, these amounts being expressed in enzyme units as determined in the paragraph above.

Preferably, the total amount of said at least one endopeptidase used in the method according to the invention is between 10 U and 500 U, more preferably, between 20 U and 150 U, still more preferably, between 30 U and 60 U, for example such as 40 U, per gram of insect cuticles, on a dry weight basis.

Advantageously, the ratio of the amount of the at least one endopeptidase used in the first U-hydrolysis per gram of insect cuticles on a dry weight basis/amount of the at least one endopeptidase used in the second U-hydrolysis per gram of insect cuticles on a dry weight basis is comprised between 0.5 and 10, preferably between 0.8 and 5, more preferably between 1 and 2, such as about 1.

Advantageously, the temperature of each enzymatic hydrolysis is between 35 and 70° C., preferably between 45 and 60° C.

Table 2 summarizes the origin and enzyme activities observed for the marketed enzymes of Table 1. Moreover, the particularly preferred enzymatic hydrolysis temperature associated with each enzyme when used in the method according to the invention is indicated.

TABLE 2

| | Enzyme activity | Preferred |
|---|---|---|
| | (U/g or U/mL | temperature |
| Enzyme | of enzyme) | (° C.) |
| Promod 439L | 588.4 | 57 |
| Novo-Pro D | 1771.8 | 60 |
| Novozym 37071 | 2031 | 60 |
| Alcalase 2.5 L PF | 908.1 | 55 |
| Corolase 7089 | 1052.3 | 55 |
| Sumizyme BNP-L | 1152 | 55 |
| Food Pro PNL | 473.5 | 50 |
| Protin SD-NY100 | 4248.5 | 50 |
| Corolase 2TS | 918.1 | 60 |
| Collagenase Type I | 522.3 | 40 |
| Promod 648L | 577.4 | 60 |
| Prolyve NP conc | 1020.7 | 45 |
| FoodPro 51FP | 751.7 | 50 |
| Sumizyme LP | 372.2 | 45 |
| Protease type XIV | 5098.6 | 40 |
| Pancreatin | 1236.4 | 45 |
| Bromelain (stem) | 851.7 | 45 |
| Lypaïne 48000 | 734.1 | 60 |

Advantageously, the enzymatic hydrolysis steps of the method according to the invention are carried out in the presence of water in a ratio of water in g/cuticles in g and on a dry weight basis comprised between 5 and 50, preferably between 10 and 40, more preferably between 15 and 30, still more preferably between 20 and 25, for example such as of the order of 22.

Advantageously, the pH of the enzymatic hydrolysis steps is between 6 and 9, preferably between 6.5 and 7.5, more preferably between 6.8 and 7.3.

Advantageously, the method according to the invention further comprises a step of collecting the hydrolyzed cuticles resulting from the second enzymatic hydrolysis.

Preferably, this collecting step is carried out by centrifugation, decantation or filtration, more preferably, by filtration.

Preferably, this filtration step is carried out using a Büchner filter, a filter press, a band filter or a sieve filter, more preferably using a Büchner filter with a metal mesh.

Preferably, the pore size or mesh size of the filter used in this filtration is between 4 μm and 800 μm, more preferably, between 40 μm and 550 μm, still more preferably between 140 μm and 530 μm.

The invention is therefore more particularly directed to a particular method for obtaining chitin from insect cuticles comprising:

a step of separating the cuticles from the soft part of the insects,
a step of grinding the insect cuticles,
a first enzymatic hydrolysis of the ground insect cuticles using at least one endopeptidase,
separating the hydrolyzed cuticles resulting from the first enzymatic hydrolysis, from the hydrolysis medium,
a second enzymatic hydrolysis of the hydrolyzed cuticles using at least one endopeptidase, excluding exopeptidase,
a thermal inactivation of said at least one endopeptidase used,
a step of collecting the hydrolyzed cuticles resulting from the second enzymatic hydrolysis

The steps of this particular method are advantageously as described above, including the embodiments. In particular, the at least one endopeptidase used in the first hydrolysis is a serine endopeptidase, a metalloendoprotease or a mixture of a serine endopeptidase, an aspartic protease and an aminopeptidase, and the at least one endopeptidase used in the second hydrolysis is in particular a serine endopeptidase or a metalloendoprotease.

The hydrolyzed cuticles resulting from the second enzymatic hydrolysis are of chitin. However, the purity of this chitin may be unsatisfactory for certain applications.

Therefore, to obtain a purified chitin, it may be advantageous for the method according to the invention to further comprise, after the second hydrolysis, a chemical treatment step.

Advantageously, the chemical treatment is carried out with a strong base.

Advantageously, the strong base is selected from sodium hydroxide or soda, potassium hydroxide and ammonium hydroxide. Preferably, the strong base is sodium or potassium hydroxide, more preferably sodium hydroxide.

Preferably, the base used for the chemical treatment is in the form of an aqueous basic solution. In this case, the molar concentration of the base in aqueous solution is advantageously between 0.1 and 4 mol.L⁻¹ , preferably between 0.5 and 2 mol.L⁻¹ , still more preferably equal to 1 mol.L⁻¹.

The chemical treatment is advantageously carried out for a time between 5 and 60 hours, preferably for a time between 15 and 25 hours, for example such as 19 hours.

Thus, by making it possible to reduce the residual amount of proteins on the surface of the hydrolyzed cuticles, the method according to the invention also has the advantage of reducing the chemical treatment time.

Advantageously, the basic treatment is carried out at a temperature comprised between 70 and 120° C., preferably between 80 and 100° C., more preferably of the order of 90° C.

Advantageously, the method according to the invention further comprises a step of collecting the purified chitin resulting from the chemical treatment step. Preferably, this collecting step is carried out by centrifugation, decantation or filtration, more preferably, by filtration.

Preferably, this filtration step is carried out using a Büchner filter, a filter press, a band filter or a sieve filter, more preferably, using a sieve filter.

Preferably, the pore size or mesh size of the filter used in this filtration is between 4 μm and 800 μm, more preferably, between 40 μm and 550 μm, still more preferably, between 140 μm and 530 μm.

Optionally, the method according to the invention further comprises a step of washing the purified chitin.

This optional step of washing the purified chitin is advantageously carried out with tap water, preferably lukewarm, i.e. having a temperature comprised between 15 and 60° C.

Preferably, the washing of the purified chitin is carried out until the pH is neutralized.

Optionally, the method according to the invention further comprises a step of drying the purified chitin.

This optional step of drying the purified chitin is advantageously performed at a temperature comprised between 40 and 105° C., preferably at about 60° C.

The drying step of the purified chitin is advantageously carried out for a time comprised between 10 and 80 hours, preferably for about 24 hours.

Advantageously, the drying of the purified chitin is carried out using a drying oven, such as the FED 115 or FP53 model from the company Binder®.

The invention also relates to a method for obtaining chitosan comprising a method for obtaining chitin according to the invention, as set forth above, and further comprising a step of deacetylating the chitin.

Advantageously, the deacetylation of the chitin is performed with a strong base.

Preferably, the strong base is selected from sodium hydroxide or soda, potassium hydroxide and ammonium hydroxide. More preferably, the strong base is sodium or potassium hydroxide, still more preferably sodium hydroxide.

Preferably, the base used for the basic treatment is in the form of an aqueous basic solution, preferably a concentrated aqueous basic solution.

In this case, the molar concentration of the base in aqueous solution is advantageously comprised between 6 and 25 mol.L⁻¹ , preferably comprised between 8 and 19 mol.L⁻¹ , more preferably comprised between 10 and 19 mol.L⁻¹ , still more preferably comprised between 12 and 19 mol.L⁻¹.

Preferably, deacetylation is performed for 1 to 24 hours and preferably 2 to 18 hours.

Advantageously, this deacetylation is carried out in two stages, with an intermediate pH neutralization step. For example, the deacetylation may be carried out in two times two hours, i.e. for 4 hours, with an intermediate pH neutralization step.

The deacetylation temperature is advantageously between 80 and 150° C., preferably between 90 and 120° C. and more preferably at 100° C.

Advantageously, the method for obtaining chitosan according to the invention further comprises a chitosan collecting step. Preferably, this collecting step is carried out by centrifugation, decantation or filtration, more preferably, by filtration.

Preferably, this filtration step is carried out using a Büchner filter, a filter press, a band filter or a sieve filter, more preferably, using a sieve filter.

Preferably, the pore size or mesh size of the filter used in this filtration is between 4 μm and 800 μm, more preferably, between 40 μm and 550 μm, still more preferably between 140 μm and 530 μm.

Optionally, the method for obtaining chitosan according to the invention further comprises a step of washing the chitosan.

This optional step of washing the chitosan is advantageously carried out with tap water, preferably lukewarm, i.e. having a temperature comprised between 15 and 60° C.

Preferably, the washing of the chitosan is carried out until the pH is neutralized.

Optionally, the method for obtaining chitosan according to the invention further comprises a step of drying the chitosan.

This optional step of drying the chitosan is advantageously carried out at a temperature comprised between 40 and 105° C., preferably at about 60° C.

The chitosan drying step is advantageously carried out for a period comprised between 10 and 80 hours, preferably for about 24 hours.

Advantageously, the drying of the chitosan is carried out using a drying oven, such as the FED 115 or FP53 model from the company Binder®.

The present invention is illustrated, in a non-limiting way, by the following examples and figures:

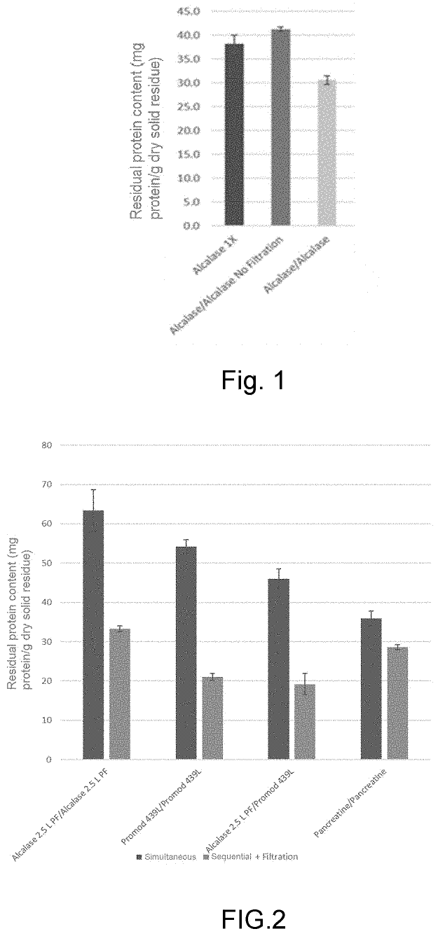

FIG. 1 is a diagram representing the residual protein content of Tenebrio molitor cuticles (in milligrams of protein per gram of solid residue) as a function of three different extraction methods using enzymatic hydrolysis with Alcalase 2.5 L PF.

FIG. 2 is a diagram representing the residual protein content of Tenebrio molitor cuticles (in milligrams of protein per gram of dry solid residue) as a function of two different extraction methods (simultaneous or sequential+filtration) using enzymatic hydrolysis with different serine endopeptidases.

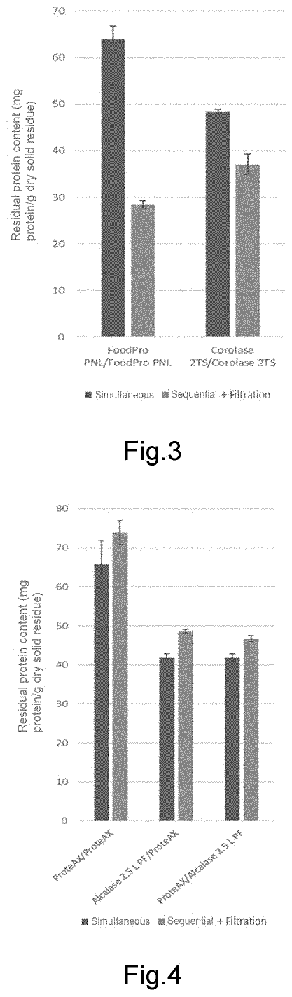

FIG. 3 is a diagram representing the residual protein content of Tenebrio molitor cuticles (in milligrams of protein per gram of dry solid residue) according to two different extraction methods (simultaneous or sequential+filtration) using enzymatic hydrolysis with different metalloendopeptidases.

FIG. 4 is a diagram representing the residual protein content of Tenebrio molitor cuticles (in milligrams of protein per gram of dry solid residue) according to two different extraction methods (simultaneous or sequential+filtration) implementing enzymatic hydrolysis with a protease comprising only an exopeptidase (ProteAX) and/or a protease comprising only a serine endopeptidase (Alcalase 2.5 L PF).

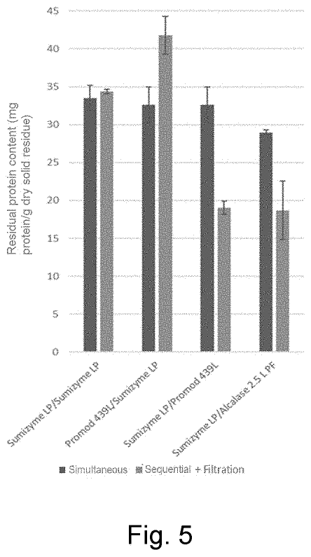

FIG. 5 is a diagram representing the residual protein content of Tenebrio molitor cuticles (in milligrams of protein per gram of dry solid residue) according to two different extraction methods (simultaneous or sequential+filtration) using enzymatic hydrolysis with a protease comprising a mixture of endopeptidases and an exopeptidase (Sumizyme LP) and/or a protease comprising only a serine endopeptidase (Promod 439L or Alcalase 2.5 L PF).

### EXAMPLE 1: OBTAINING GROUND INSECT CUTICLES

Live larvae of Tenebrio molitor, having received a diet based on cereal coproducts (wheat bran type), are conveyed in a layer of thickness comprised between 2 and 10 cm, on a belt with perforations (1 mm) to a blanching chamber. The larvae are then blanched with water at 92° C. (spray nozzles) for 5 min. The temperature of the larvae after blanching is comprised between 75° C. and 92° C.

After blanching, the larvae are conveyed to the feed hopper of a belt separator (belt separator 601 from Baader), in order to separate the cuticles from the soft part of the larvae. The diameter of the drum perforations is 1.3 mm. The separation is carried out immediately after the killing so that the larvae do not have time to cool to room temperature. The soft part of the insects is collected in a tank and the cuticles are collected using a scraper blade.

The cuticles are then ground in the presence of water (in a weight ratio of cuticles to water of 1:6, for example 1200 mL water to 200 g cuticles) using a Thermomix Vorwerk for 1 minute at speed 10, leading to ground cuticles with a size of about 1-2 mm. They are then filtered on a Büchner with a metal filter with a mesh size of 500 μm, and washed with water (same volume of water as used for grinding, i.e. 1200 mL water in this example).

Lastly, the washed cuticles are pressed (Angelia 7500, Angel) to remove excess water so as to obtain a dry matter content of 52% ±5 by weight.

### Example 2: Effect of Filtration between Two Enzymatic Extractions with a Protease of Bacterial Origin—Alcalase 2.5 L PF

Alcalase 2.5 L PF is a serine endopeptidase derived from Bacillus licheniformis and marketed by Novozymes.

A total of 260 U of Alcalase 2.5 L PF is added to a mixture of 8.6 g of ground cuticles (wet weight) obtained according to the method described in Example 1, and 86 g of tap water, using three different methods. The pH of the reaction medium before the addition of protease in these three methods is 7.3. The three methods are carried out at a temperature of 55° C. and the results are shown in FIG. 1:

“Alcalase 1X”: the 260 U Alcalase are added in a single time and the hydrolysis reaction is left for 5 h.
“Alcalase/Alcalase No filtration”: 130 U Alcalase are added, the first hydrolysis is left for 2 h, then 130 U Alcalase are added again and the second hydrolysis is left for 3 h.
“Alcalase/Alcalase”: 130 U Alcalase are added, the first hydrolysis is left for 2 h and the reaction medium is then filtered (Büchner filter with a 160 μm metal mesh). The recovered cuticles are then mixed with 86 g tap water and 130 U Alcalase are added again, then the second hydrolysis is left for 3 h.

After inactivation of the enzyme for 15 min at 85° C., the reaction media are filtered (Büchner filter with a 160 μm metal mesh).

The residual protein content is determined by the BCA (BiCinchoninic acid Assay) method, using 2 mg of dry solid residue. The reaction medium obtained from the BCA method is diluted with water with a dilution factor of 4 prior to spectrophotometer measurement.

Thus, for a same total amount of added protease, deproteinization is slightly less efficient when two hydrolyses are carried out successively and without filtration compared to a single hydrolysis (respectively 41 mg and 38 mg of residual protein per gram of solid residue).

However, when two hydrolyses are carried out successively and with filtration between the two hydrolyses, the degree of deproteinization is significantly increased compared to the hydrolysis performed in a single time (31 mg and 38 mg of residual protein per gram of solid residue, respectively).

Therefore, a filtration step between two successive hydrolyses has the effect of increasing the degree of deproteinization of insect cuticles, and thus the purity of the resulting chitin.

Example 3: Method According to the Invention and Comparative Method—serine endopeptidase

The serine endopeptidases used in this Example 3 are subtilisin and pancreatin. The subtilisin is from two different suppliers: Alcalase 2.5 L PF (Novozymes) and Promod 439L (Biocatalysts), and is derived from Bacillus licheniformis. The pancreatin is from Sigma-Aldrich and is derived from Sus scrofa domesticus.

Two different methods were used: simultaneous (comparative method) and sequential+filtration (method according to the invention), the results of which are presented in FIG. 2.

Simultaneous Method

Protease is added to a mixture of 2 g of ground cuticles (dry weight), obtained according to the method described in Example 1, with 39.8 g of tap water at pH 7.3.

The simultaneous method was carried out by adding a total of 40 U of protease(s) per gram of cuticle (dry weight) to the mixture of cuticle and water, for a reaction time of 4 h and a temperature depending on the protease used.

Alcalase 2.5 L PF/Alcalase 2.5 L PF: a single hydrolysis step with 40 U of Alcalase 2.5 L PF per gram of cuticle (dry weight) and at a temperature of 55° C.

Promod 439 L/Promod 439 L: a single hydrolysis step with 40 U Promod 439 L per gram of cuticles (dry weight) and at a temperature of 57° C.

Alcalase 2.5 L PF/Promod 439 L: a single hydrolysis step with 20 U of Alcalase 2.5 L PF and with 20 U of Promod 439 L per gram of cuticles (dry weight), and at a temperature of 56° C.

Pancreatin/Pancreatin: a single hydrolysis step with 40 U of Pancreatin per gram of cuticle (dry weight) and at a temperature of 45° C.

Sequential Method+Filtration

Protease is added to a mixture of 2 g of ground and washed cuticles (dry weight), obtained according to the method described in Example 1, with 39.8 g of tap water at pH 7.3.

The sequential +filtration method was also performed by adding a total of 40 U of protease per gram of cuticle (dry weight) to the mixture of cuticle and water, for a total reaction time of 4 h and a temperature depending on the protease used. However, in this method, a first hydrolysis is performed with 20 U of protease per gram of cuticles (dry weight) for 2 h, then the hydrolysis medium is filtered (160 μm mesh); the recovered hydrolyzed cuticles are then mixed again with 39.8 g of tap water at a pH of 7.3 and a second hydrolysis is again performed with 20 U of protease per gram of cuticles (dry weight) for 2 h.

Alcalase 2.5 L PF/Alcalase 2.5 L PF: first hydrolysis with 20 U of Alcalase 2.5 L PF per gram of cuticle (dry weight) at a temperature of 55° C., then filtration of the hydrolysis medium, and second hydrolysis with 20 U of Alcalase 2.5 L PF per gram of cuticle (dry weight) at a temperature of 55° C. Promod 439L/Promod 439L: first hydrolysis with 20 U of Promod 439 L per gram of cuticles (dry weight) at a temperature of 57° C., then filtration of the hydrolysis medium, and second hydrolysis with 20 U of Promod 439 L per gram of cuticles (dry weight) at a temperature of 57° C.

Alcalase 2.5 L PF/Promod 439 L: first hydrolysis with 20 U Alcalase 2.5 L PF per gram of cuticles (dry weight) at a temperature of 55° C., then filtration of the hydrolysis medium, and second hydrolysis with 20 U of Promod 439 L per gram of cuticles (dry weight) at a temperature of 57° C.

Pancreatin/Pancreatin: first hydrolysis with 20 U Pancreatin per gram of cuticle (dry weight) at a temperature of 45° C., then filtration of the hydrolysis medium, and second hydrolysis with 20 U Pancreatin per gram of cuticle (dry weight) at a temperature of 45° C.

In both methods (simultaneous or sequential+filtration), the protease is then inactivated for 15 min at 85° C., and the reaction medium is filtered (160 μm mesh size).

The residual protein content is determined by the BCA (BiCinchoninic acid Assay) method, using 2 mg of dry solid residue. The reaction medium obtained from the BCA method is diluted with water with a dilution factor of 4 prior to spectrophotometer measurement.

FIG. 2 shows that when two successive hydrolyses are performed with the protease and with filtration between the two hydrolyses (sequential+filtration), the degree of deproteinization is significantly increased compared to hydrolysis performed in a single time (simultaneous), this being for the three proteases tested (see Alcalase 2.5 L PF/Alcalase 2.5 L PF, Promod 439 L/Promod 439 L and Pancreatin/Pancreatin), or for alternating proteases (see Alcalase 2.5 L PF/Promod 439 L).

Therefore, a filtration step between two hydrolyses with a serine endopeptidase has the effect of significantly increasing the degree of deproteinization of the insect cuticles, and thus the purity of the resulting chitin.

### Example 4: Method According to the Invention and Comparative Method—metalloendopeptidase

The metalloendopeptidases used in this Example 4 are bacillolysin and thermolysin. The bacillolysin is FoodPro PNL which is from Dupont Danisco and is derived from Bacillus amyloliquefaciens. The thermolysin is Corolase 2TS from AB Enzymes and is derived from Bacillus stearothermophilus.

The simultaneous and sequential+filtration methods of Example 3 were replicated with these metalloendopeptidases and at a temperature of 50° C. for FoodPro PNL and 60° C. for Corolase 2TS. The results are presented in FIG. 3.

FIG. 3 shows that when two successive hydrolyses are performed with FoodPro PNL or Corolase 2TS and with filtration between the two hydrolyses (sequential+filtration), the degree of deproteinization is significantly increased compared to hydrolysis performed in a single time (simultaneous).

Therefore, a filtration step between two hydrolyses with a metalloendopeptidase has the effect of significantly increasing the degree of deproteinization of insect cuticles, and thus the purity of the resulting chitin.

### Example 5: Influence of the Nature of the Protease: Requirement for the Presence of an endopeptidase in each Hydrolysis Step

In order to study the influence of the endo- or exopeptidase nature on the degree of deproteinization, a protease comprising mainly aminopeptidase (exopeptidase) was used (ProteAX derived from Aspergillus oryzae and sold by Amano Enzyme; IUB number stated by the supplier: 3.4.11.1), as well as subtilisin (Alcalase 2.5 L PF).

The simultaneous and sequential+filtration methods of Example 3 were replicated, with the results shown in FIG. 4:
ProteAX/ProteAX: the temperature is 50° C.
Alcalase 2.5 L PF/ProteAX: for the simultaneous method, the temperature is 52.5° C.; for the sequential method, Alcalase 2.5 L PF is added first at a temperature of 55° C., and ProteAX is added second at a temperature of 50° C.
ProteAX/Alcalase 2.5 L PF: for the simultaneous method, the temperature is 52.5° C.; for the sequential method, ProteAX is added first at a temperature of 50° C., and Alcalase 2.5 L PF is added second at a temperature of 55° C.

FIG. 4 shows that when two successive hydrolyses are performed with ProteAX and with filtration between the two hydrolyses, the rate of deproteinization is significantly decreased compared to hydrolysis performed in a single time. Moreover, the fact that two successive hydrolyses are performed with ProteAX before or after Alcalase 2.5 L PF does not make it possible to increase the degree of deproteinization compared to a hydrolysis performed in a single time with both proteases (simultaneous addition): the use of ProteAX in a sequential method+filtration therefore significantly decreases the degree of deproteinization.

Therefore, in the method according to the invention, the presence of an endopeptidase is necessary in each hydrolysis step in order to increase the deproteinization rate.

### Example 6: Influence of the Nature of the Protease in the Second Enzymatic Hydrolysis: Requirement for a Protease not including an exopeptidase in the Second Hydrolysis Step

In order to study the influence of the use of a protease comprising both endopeptidases and an exopeptidase on the degree of deproteinization, a protease comprising a mixture of oryzin, aspergillopepsin I (endopeptidases) and leucyl aminopeptidase (exopeptidase) was used (Sumizyme LP derived from Aspergillus oryzae and sold by Shin Nihon Chemical), optionally alternating with subtilisin (Alcalase 2.5 L PF and Promod 439 L).

The simultaneous and sequential+filtration methods of Example 3 were replicated, with the results shown in FIG. 5:
Sumizyme LP/Sumizyme LP: the temperature is 45° C.
Promod 439L/Sumizyme LP: for the simultaneous method, the temperature is 50° C.; for the sequential method, Promod 439L is added first at a temperature of 57° C., and Sumizyme LP is added second at a temperature of 45° C.
Sumizyme LP/Promod 439 L: for the simultaneous method, the temperature is 50° C.; for the sequential method, Sumizyme LP is added first at a temperature of 45° C., and Promod 439 L is added second at a temperature of 57° C.
Sumizyme LP/Alcalase 2.5 L PF: for the simultaneous method, the temperature is 50° C.; for the sequential method, Sumizyme LP is added first at a temperature of 45° C., and Alcalase 2.5 L PF is added second at a temperature of 55° C.

When two successive hydrolyses are performed with Sumizyme LP and with filtration between the two hydrolyses, the degree of deproteinization is similar to the hydrolysis performed in a single time. Furthermore, a second hydrolysis with Sumizyme LP after a first hydrolysis with Promod 439 L followed by filtration significantly decreases the degree of deproteinization compared to a hydrolysis carried out in a single time.

However, if the first hydrolysis is performed with Sumizyme LP and the second with Promod 439 L in the sequential+filtration method (Sumizyme LP/Promod 439 L), then the degree of deproteinization is significantly increased compared to the hydrolysis performed at a single time. The case is similar when Promod 439L is replaced by Alcalase 2.5 L PF (Sumizyme LP/Alcalase 2.5 L PF).

Therefore, when filtration is carried out between two enzymatic hydrolyses, it is not necessary for the protease used in the first hydrolysis not to comprise exopeptidase. However, this condition is imperative for the protease used in the second hydrolysis, and allows the degree of deproteinization to be significantly increased.

1. Method for obtaining chitin from insect cuticles comprising:
performing a first enzymatic hydrolysis of insect cuticles using at least one endopeptidase,
separating the hydrolyzed cuticles resulting from the first enzymatic hydrolysis, from the hydrolysis medium,
performing a second enzymatic hydrolysis of the hydrolyzed cuticles using at least one endopeptidase, excluding exopeptidase.

2. Method according to claim 1, wherein the at least one endopeptidase used in the second enzymatic hydrolysis is an endopeptidase or a mixture of endopeptidases selected from serine endopeptidases, cysteine endopeptidases, aspartic proteases, metalloendopeptidases and threonine endopeptidases.

3. Method according to claim 1, wherein the at least one endopeptidase used in the second enzymatic hydrolysis is a serine endopeptidase or a mixture of serine endopeptidases, and/or a metalloendoprotease or a mixture of metalloendoproteases, each serine endopeptidase being selected from the group constituted by subtilisin and trypsin, each metalloendoprotease being selected from the group constituted by bacillolysin and thermolysin.

4. Method according to claim 1, wherein the at least one endopeptidase used in the second enzymatic hydrolysis is of bacterial origin.

5. Method according to claim 1, further comprising a step of grinding the insect cuticles prior to the first enzymatic hydrolysis.

6. Method according to claim 1, wherein the insect cuticles are obtained by a step of separating the cuticles from the soft part of the insects.

7. Method according to claim 1, wherein the total time of the hydrolysis steps is less than 6 hours.

8. Method according to claim 1, wherein the total amount of said at least one endopeptidase used is between 10 U and 500 U per gram of insect cuticle, on a dry weight basis.

9. Method according to claim 1, further comprising, after the second hydrolysis, a chemical treatment step.

10. Method of obtaining chitosan comprising a method of obtaining chitin according to claim 1, and further comprising a step of deacetylating the chitin.
